(19) United States
(12) Patent Application Publication
(10) Pub. No.: US 2009/0068753 A1
(43) Pub. Date: Mar. 12, 2009

(54) Detection of bromamines and chloramines

(76) Inventors: Somasuntharam Tharmalingam Senthilmohan, Christchurch (NZ); Murray James McEwan, Christchurch (NZ)

Correspondence Address: D. Peter Hochberg;D. Peter Hochberg Co., 1940 East 6th St. - 6th Floor, Cleveland, OH, 44114, US

(21) Appl. No.: 11/665,924

(22) PCT Filed: Oct. 18, 2005
(86) PCT No.: PCT/NZ05/00271

§ 371 Date: Apr. 20, 2007

(30) Foreign Application Priority Data
Oct. 22, 2004 (NZ) 536082

(51) Int. Cl. G01N 33/50

(52) U.S. Cl. 436/111

(57) ABSTRACT

A method for the detection of chloramines and bromamines which are indicative markers particularly of lung diseases using flow tube, drift tube or atmospheric chemical ionisation technologies. The method can also be utilised to detect oxidative stress related diseases, test the prophylactic effect of drugs given to a patient, and to detect the level of chloramines in swimming pools and like situations.

### CROSS-REFERENCE TO RELATED APPLICATION

This application is a National Stage application of International Application No. PCT/NZ2005/000271, filed on Oct. 18, 2005, which claims priority of New Zealand application No. 536082 filed on Oct. 22, 2004.

### BACKGROUND OF THE INVENTION

Many lung diseases, including asthma, chronic obstructive pulmonary disease (COPD), bronchiectasis, cystic fibrosis, and interstitial lung disease, involve chronic inflammation and oxidative stress. The lung diseases cause airflow blockage and breathing-related problems.

Asthma is a respiratory disease which is often due to an allergy. The common asthma allergies include dust, animal fur or feathers, moulds, and pollen. Currently, asthma diagnosis and the level of severity are judged purely on the symptoms a patient suffers. However, over the past decade researchers have discovered that symptoms alone do not always give a very good indication of the extent of the condition. (Kharitonov et al. 2001; Am J Respir Crit Care Med. 163; 1693-1722)

Government figures show asthma affects more than 20 million people in the United States and accounts for nearly 5,000 deaths each year.

COPD encompasses both chronic bronchitis and emphysema and is one of the commonest respiratory conditions of adults in the developed world. COPD poses an enormous burden to society both in terms of direct cost to healthcare services and indirect costs to society through loss of productivity. The annual cost to the U.S. for COPD is $32.1 billion

COPD is a leading cause of death, illness, and disability. While it is recognized that tobacco use is a key factor in the development and progression of COPD, exposure to air pollutants in the home and workplace, genetic factors, and respiratory infections also play a role. In the developing world, indoor air quality is thought to play a large role in the development and progression of COPD.

It has been estimated for instance that in the United States, up to 24 million people are affected by COPD and 118,000 people died in 2001.

In the detection of asthma, fiber optic bronchial biopsies have become the “gold standard” for measuring inflammation in the airway wall, but this is an invasive procedure that is not suitable for routine clinical practice and cannot be repeated often. The second method is measurement of airway hyper responsiveness by histamine or methacholine challenge which has been used as a surrogate marker of inflammation. It involves inhalation of hypertonic saline, which may induce coughing and bronchoconstriction (Parameswaran et al. 2000; Eur Respir J. 15; 486-490). Both methods are unsuitable for use in children and patients with severe disease (Kharitonov et al. 2001; Am J Respir Crit. Care Med. 163; 1693-1722). Nitric oxide (NO) is the most extensively studied exhaled marker and abnormalities in exhaled NO have been demonstrated in several lung diseases (Kharitonov et al. 2000; Eur Respir J. 16; 781-792), particularly asthma (Gustafsson et al. 1998; Eur Respir J. Suppl. 26; 49S-52S., Kharitonov et al. 1999; Eur Respir J. 9; 212-218, and Kharitonov et al. 2000; Eur Respir J. 16; 781-792). It was reported that NO is derived from airways rather than from alveoli (Persson et al. 1993; Am Rev Respir Dis. 148; 1210-1214 & Kharitonov et al. 1996; Am J Respir Crit. Care Med. 153; 1773-1780). Exhaled NO is therefore most likely to be of epithelial rather than of endothelial origin. NO alone may not represent a sufficiently robust marker of airway inflammation. (Gibson et al. 2000; Eur Respir J. 26; 271-276).

Early detection of asthma & COPD might alter its course and progress but to date there has been no simple, reliable test that can detect both asthma & COPD in its early stages. Treatment of COPD and asthma requires a careful and thorough evaluation by a physician and prior to the present invention, the level of severity of each disease has been judged purely on the symptoms a patient suffers. However, it has been noted that symptoms alone do not always give a very good indication of the extent of the condition.

It is therefore apparent that a rapid, real time accurate diagnosis of lung diseases and in particular asthma and COPD will be of great benefit particularly in the early detection of the disease.

Research has revealed that breath samples from patients that contain trace volatiles of bromamine and chloramine compounds are indicative markers of asthma and COPD.

### SUMMARY OF THE INVENTION

It is therefore an object of this invention to provide a rapid method for detection of bromamines and chloramines.

It is a further object of the invention to provide a means for the rapid identification of asthma and COPD and lung diseases using indicative markers in breath samples.

According to one aspect the invention is a method of detecting chloramines and bromamines using flow tube, drift tube or atmospheric chemical ionisation technologies comprising the steps of:

injecting individual breaths of a patient into a stream of helium or a mixture of inert gases containing precursor ions,

ionizing by means of flow tube, drift tube or atmospheric chemical ionisation methodology the reactive metabolites in the stream to form product ions

determining the ratio of product ions to precursor ions

analyzing each trace gas constituent and

comparing the level of each gas constituent against a predetermined level.

Preferably the precursor ions are H₃O⁺, NO⁺ or O₂⁺.

Preferably the inert gas or a mixture of inert gases contain negative precursor ions.

In another aspect the method of the invention comprises the detection of lung diseases by injecting individual breaths of a patient into a stream of helium or a mixture of inert gas or gases containing precursor ions,

ionizing by means of flow tube, drift tube or atmospheric chemical ionisation methodology the reactive metabolites in the stream to form product ions

determining the ratio of product ions to precursor ions

analyzing each trace gas constituent

measuring the amount of each gas constituent and

comparing the level of each gas constituent against a predetermined level to determine the level of bromamines or chloramines in the breath of the patient

Preferably the disease to be detected is COPD.

Preferably the disease to be detected is asthma.

Preferably the disease to be detected is an oxidative stress related disease. Preferably the method of the invention is utilised to test the prophylactic effect of drugs given to a patient.

In another aspect, the invention is a method of detecting chloramines or bromamines comprising the steps of:

introducing a sample of chloramine or bromamine into a stream of an inert gas or a mixture of inert gases containing precursor ions,

ionizing the reactive metabolites in the stream to form product ions

determining the ratio of product ions to precursor ions

analyzing each trace gas constituent and

measuring the amount of each gas constituent by flow tube, drift tube or atmospheric ionization methodologies, and

comparing the level of each gas constituent against a predetermined level.

Preferably the precursor ions are H₃O⁺, NO⁺ or O₂⁺.

Preferably the inert gas or a mixture of inert gases contain negative precursor ions.

Preferably the inert gas is helium.

### BRIEF DESCRIPTION OF THE DRAWINGS

In the accompanying drawings, the graphs show the results of tests using Selective Ion Flow Tube Mass Spectrometry (SIFT-MS).

FIG. 1 is a measure of an individual real-time breath sample of bromamine in several individuals using SIFT-MS.

FIG. 2 is a measure of an individual real-time breath sample of chloramine in several individuals using SIFT-MS.

FIG. 3 is a measure of a real-time prepared bromamine standard sample using SIFT-MS.

FIG. 4 is a measure of a real-time prepared chloramine standard sample using SIFT-MS.

### DETAILED DESCRIPTION OF THE PREFERRED EMBODIMENTS

Human breath is a good example of a mixture of trace volatile species. Some of the trace volatiles are pathologically important and can be used as potential breath markers. The trace metabolites, chloramines and bromamines have been found to act as markers for lung diseases such as asthma disease and COPD in patients.

Breath samples from asthmatics, COPD subjects and healthy volunteers were tested for bromamine and chloramine compounds. Diseased subjects and healthy volunteers have shown a distinctive pattern in bromamine and chloramine compounds. As a result of the tests it has been found that, monobromamine (BrNH₂) and dibromamine (Br₂NH) are indicative markers for asthma. For COPD sufferers, monochloramine (CINH₂) and dichloramine (Cl₂NH) are indicative markers. Elevated levels of bromamine compounds were found in asthmatics compared to healthy normal subjects. In COPD subjects, elevated chloramine products were found compared to healthy normal subjects.

The trace metabolites are measured in a single breath exhalation by flow tube or drift tube methodologies or by atmospheric chemical ionization methods. In a highly preferred form of the invention, the trace metabolites were measured by selective ion flow tube mass spectrometry (SIFT-MS). In SIFT-MS methodology, an exhaled single breath is introduced into a stream of helium or a mixture of inert gases containing precursor ions. In a preferred embodiment the precursor ions are H₃O⁺, NO⁺ or O₂⁺ but the inert gas or mixture of inert gases may contain other precursor ions or negative precursor ions.

The reactive metabolites in the breath are ionised by the process of chemical ionization to form product ions of the reactive species. The ratio of the product ions to precursor ions gives the absolute concentration of the sample. The analysis of each trace gas constituent is completed and displayed within seconds.

As stated above, while the trace metabolites are preferably measured by Selective Ion Flow Tube Mass Spectrometry (SIFT-MS), it is to be understood the measurements can also be made using other flow tube or drift tube methodologies or by atmospheric chemical ionization methods.

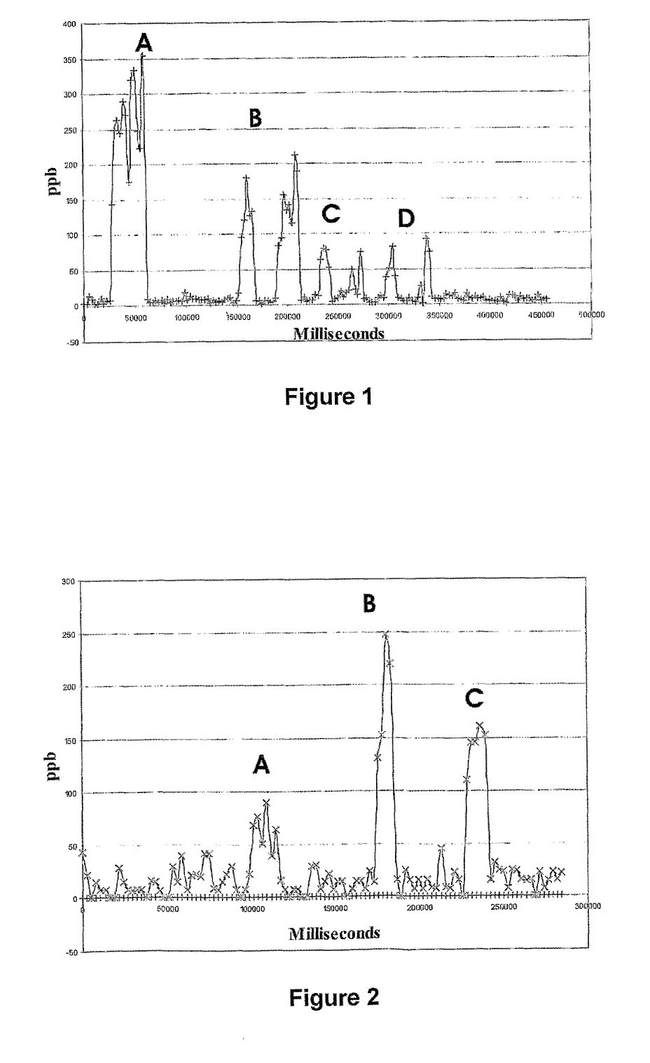

As illustrated in the FIG. 1 which is a measurement showing bromamine in individual breath samples, the subject A and B are asthmatics and the subjects C and D are healthy people.

FIG. 2 is a measurement of breath samples showing chloramines, the subject A is a healthy person and the subjects B and C suffer from COPD.

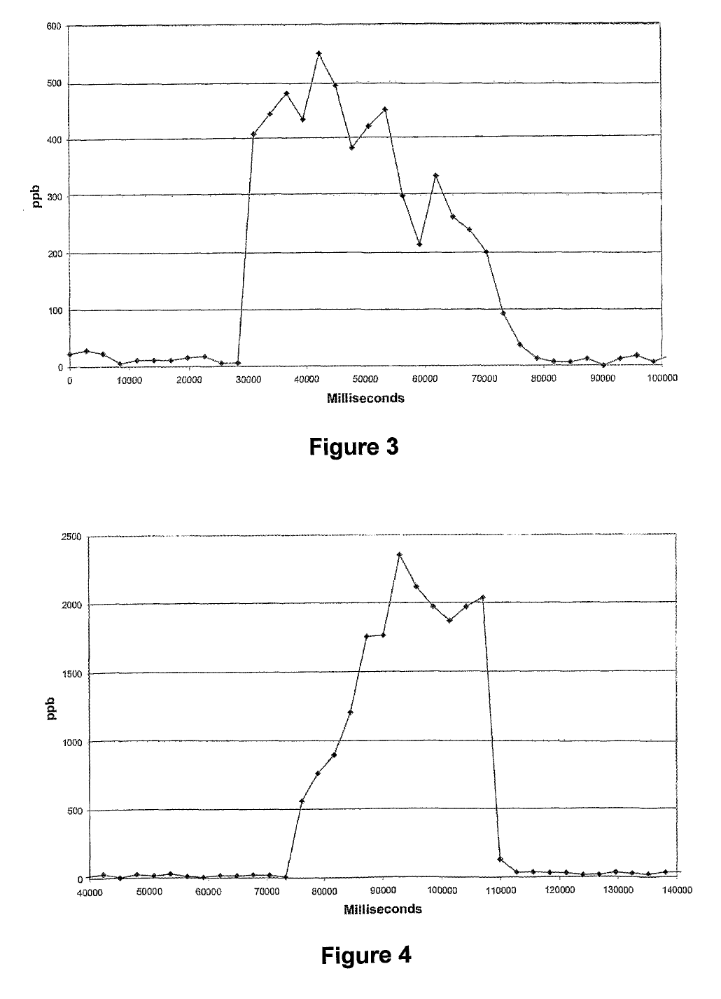

FIG. 3 is a measurement of a real-time bromamine standard sample using SIFT-MS, and

FIG. 4 is a measurement of a real-time chloramine standard sample using SIFT-MS.

Bromamine concentrations in individual expired breaths were observed to be elevated in asthmatic patients. Typical levels observed for asthmatics were between 100-300 ppb. However, in healthy normal subjects and asthmatic subjects under regular medication the levels were between 20-100 ppb respectively (FIG. 1).

Chloramine concentrations in individual breaths were observed to be elevated in COPD patients. Typical levels observed for COPD patients were between 90-250 ppb. However in healthy normal subjects the levels were between 10-80 ppb (FIG. 2).

As a result of the method of this invention it is possible to detect asthma and COPD using the observed product signals from bromamine and chloramine compounds. Monobromamine (BrNH₂) and dibromamine (Br₂NH) are found to be good markers for asthma. For COPD, monochloramine (CINH₂) and dichloramine (Cl₂NH) are found to be good markers.

Both bromamine and chloramine standards were shown in FIGS. 3 and 4 respectively.

The method of this invention uses flow tube or drift tube methodologies such as SIFT-MS to measure the marker bromamine and chloramine compounds and thus provides a rapid, inexpensive and accurate online breath measurement to identify asthma, COPD, other lung diseases and is also applicable to identify any oxidative stress related disease of humans and mammals. The method is also applicable to testing the prophylactic effect of drugs given to patients to alleviate their breathing difficulties and also to detect the levels of chloramines in swimming pools and like situations.

Oxidative stress related diseases can also be detected by utilising the method as herein described.

Having described preferred methods of putting the invention into effect, it will be apparent to those skilled in the art to which this invention relates, that modifications and amendments to various features and items can be effected and yet still come within the general concept of the invention. It is to be understood that all such modifications and amendments are intended to be included within the scope of the present invention.

1. A method of detecting chloramines and bromamines using flow tube, drift tube or atmospheric chemical ionisation technologies comprising the steps of:
injecting individual breaths of a patient into a stream of helium or a mixture of inert gases containing precursor ions;
ionizing by means of flow tube, drift tube or atmospheric chemical ionisation methodology the reactive metabolites in the stream to form product ions;
determining the ratio of product ions to precursor ions;
analyzing each trace gas constituent; and
comparing the level of each gas constituent against a predetermined level.

2. The method of claim 1 wherein the precursor ions are H₃O⁺, NO⁺ or O₂⁺.

3. The method of claim 1 wherein the inert gas helium or a mixture of inert gases contain negative precursor ions.

4. A method for the detection of lung diseases comprising injecting individual breaths of a patient into a stream of helium or a mixture of inert gas or gases containing precursor ions;
ionizing by means of flow tube, drift tube or atmospheric chemical ionisation methodology the reactive metabolites in the stream to form product ions;
determining the ratio of product ions to precursor ions;
analyzing each trace gas constituent;
measuring the amount of each gas constituent; and
comparing the level of each gas constituent against a predetermined level to determine the level of bromamines or chloramines in the breath of the patient.

5. The method of claim 4 wherein the disease to be detected is COPD.

6. The method of claim 4 wherein the disease to be detected is asthma.

7. The method of claim 4 wherein the disease to be detected is an oxidative stress related disease.

8. The method of claim 1 when wherein said method is utilised to test the prophylactic effect of drugs given to a patient.

9. A method of detecting both mono and di-chloramines or both mono and di-bromamines comprising the steps of:
introducing a sample of chloramine or bromamine into a stream of an inert gas or a mixture of inert gases containing precursor ions;
ionizing the reactive metabolites in the stream to form product ions;
determining the ratio of product ions to precursor ions;
analyzing each trace gas constituent;
measuring the amount of each gas constituent by flow tube, drift tube or atmospheric ionization methodologies; and
comparing the level of each gas constituent against a predetermined level.

10. The method of claim 9 wherein the precursor ions are H₃O⁺, NO⁺ or O₂⁺.

11. The method of claim 9 wherein the inert gas or a mixture of inert gases contain negative precursor ions

12. The method of claim 9 wherein the inert gas is helium.
